(19) United States
(12) Patent Application Publication
(10) Pub. No.: US 2018/0008183 A1
(43) Pub. Date: Jan. 11, 2018

(54) DEVICES AND METHODS FOR DELIVERING AND/OR RECEIVING FLUID

(71) Applicant: Seventh Sense Biosystems, Inc., Medford, MA (US)

(72) Inventors: Donald E. Chickering, III, Framingham, MA (US); Li Yang Chu, Medford, MA (US); Sergey Grigoryants, Malden, MA (US); Scott James, Epping, NH (US); Jacob Wachlin, Cambridge, MA (US)

(73) Assignee: Seventh Sense Biosystems, Inc., Medford, MA (US)

(21) Appl. No.: 15/545,380

(22) PCT Filed: Jan. 28, 2016
(86) PCT No.: PCT/US2016/015259

§ 371 Date: Jul. 21, 2017

Related U.S. Application Data
(60) Provisional application No. 62/108,653, filed on Jan. 28, 2015.

(51) Int. Cl. A61B 5/15; A61B 5/157

(52) U.S. Cl. CPC A61B 5/150946; A61B 5/150862; A61B 5/150267; A61B 5/157; A61B 5/150335; A61B 5/150099; A61B 2560/0214

(57) ABSTRACT

The present invention generally relates to collecting, determining and/or transmitting information associated with the process of receiving bodily fluid through a device opening and/or delivering substances to a subject. In one aspect, the device includes an information collecting portion and a fluid receiving portion. In another aspect, the two portions are constructed and designed to attach to one another. In one embodiment, actuation of one of the portions causes the other portion to actuate. Methods of time keeping and information transmission are also discussed.

### RELATED APPLICATIONS

This application claims the benefit of U.S. Provisional Patent Application Ser. No. 62/108,653, filed Jan. 28, 2015, incorporated herein by reference.

### FIELD OF INVENTION

The present invention generally relates to systems and methods for collecting, determining and transmitting information associated with the process of delivering to and/or receiving fluids or other materials, such as blood or interstitial fluid, from subjects, e.g., to or from the skin and/or beneath the skin.

### BACKGROUND

Phlebotomy or venipuncture is the process of obtaining intravenous access for the purpose of intravenous therapy or obtaining a sample of venous blood. This process is typically practiced by medical practitioners, including paramedics, phlebotomists, doctors, nurses, and the like. Substantial equipment is needed to obtain blood from a subject, including the use of evacuated (vacuum) tubes, e.g., such as the VACUTAINER (Becton, Dickinson and Company) and VACUETTE (Greiner Bio-One GmBH) systems. Other equipment includes hypodermic needles, syringes, and the like. However, such procedures are complicated and require sophisticated training of practitioners, and often cannot be done in non-medical settings. Accordingly, improvements in methods of obtaining blood or other fluids from or through the skin are still needed.

### SUMMARY OF INVENTION

In some embodiments, the present invention generally relates to devices and methods for receiving fluids from a subject, such as the reception and separation of blood to form plasma or serum. The subject matter of the present invention involves, in some cases, interrelated products, alternative solutions to a particular problem, and/or a plurality of different uses of one or more systems and/or articles.

According to one aspect, a device for receiving fluid from a subject is provided. The device includes a device actuator and a fluid receiving portion constructed and designed to receive fluid from the subject. The fluid receiving portion includes a fluid storage chamber for storing the fluid released by the subject. The device also includes an information collecting portion that is constructed and designed to attach to the fluid receiving portion. The information collecting portion is constructed and designed to collect information associated with operation of the fluid receiving portion or the fluid stored in the storage chamber. Activation of the device actuator actuates the fluid receiving portion to begin a fluid collecting process and actuates the information collecting portion to collect information.

According to another aspect, an information collecting device for collecting information associated with release of fluid from a subject or receipt of fluid into a medical device is provided. The information collecting device includes a housing that is constructed and designed to attach to a fluid receiving device having a fluid storage chamber for storing the fluid released by the subject. The information collecting device also includes a controller constructed and designed to collect information associated with operation of the fluid receiving device or fluid stored in the storage chamber of the fluid receiving device. The information collecting portion is actuated to collect information when the fluid receiving device undergoes a fluid collecting process.

According to another aspect, a method of determining a time at which a fluid receiving portion is actuated to cause fluid to be released from a subject is provided. The method includes receiving fluid into a fluid receiving device in response to activation of a device actuator. The method also includes actuating a controller of an information collecting portion to begin counting time in response to the activation of the device actuator. The method also includes transmitting time information from the controller to a reader. The information collecting portion and the fluid receiving device are constructed and designed to attach to one another.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments that incorporate one or more aspects of the invention will be described by way of example with reference to the accompanying figures, which are schematic and are not necessarily intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a top perspective view of a device having a fluid receiving portion and an information collecting portion in accordance with aspects of the invention;

FIG. 2 is a top perspective view of the device shown in FIG. 1 with the cover of the housing of the information collecting portion shown in phantom to reveal the fluid receiving portion beneath;

FIG. 3 is a top perspective view of a device having a fluid receiving portion separated from an information collecting portion in accordance with aspects of the invention;

FIG. 4 is a top perspective view of the device shown in FIG. 3 with the cover of the information collecting portion shown in phantom;

FIG. 5 is a perspective view of a device with a fluid receiving portion attached to an information receiving portion in accordance with aspects of the invention, where the information receiving portion is shown in cross-section;

FIG. 6 is a top perspective view of an information collecting portion;

FIG. 7 is a top perspective view of the information collecting portion shown in FIG. 6 with the cover shown in phantom;

FIG. 8 is a top perspective view of the information collecting portion shown in FIG. 6 with the cover removed;

FIG. 9 is a side view of the information collecting portion shown in FIG. 6 with the cover shown in phantom;

FIG. 10 is a side view of the information collecting portion shown in FIG. 6 with the cover removed;

FIG. 11 is a bottom perspective view of the information collecting portion shown in FIG. 6;

FIG. 12 is a bottom perspective view of the information collecting portion shown in FIG. 6 with the bottom support shown in phantom;

FIG. 13 is a bottom perspective view of the information collecting portion shown in FIG. 6 with the bottom support removed;

FIG. 14 is a top perspective exploded view of the information collecting portion shown in FIG. 6;

FIG. 15 is a bottom perspective exploded view of the information collecting portion shown in FIG. 6;

FIG. 16A is a perspective view of a device prior to actuation, where the information collecting device is shown in cross-section; and

FIG. 16B shows the arrangement shown in FIG. 16A during actuation.

### DETAILED DESCRIPTION

Aspects of the invention are not limited in application to the details of construction and the arrangement of components set forth in the following description or illustrated in the drawings. For example, illustrative embodiments relating to piercing skin and receiving blood released from the pierced skin are discussed below, but aspects of the invention are not limited to use with devices that pierce skin and/or receive blood. Other embodiments may be employed, such as devices that receive other bodily fluids without piercing, and aspects of the inventions may be practiced or be carried out in various ways. Also, the phraseology and terminology used herein is for the purpose of description and should not be regarded as limiting.

With conventional fluid collection devices, a user refers to a clock and manually records the date and time at which a sample is taken. The device itself does not record the time and date at which the sample is taken. The inventors have appreciated that manual time records may be misplaced, inaccurate, delayed in time from the actual administration or collection, or the user may forget to record the time altogether. The inventors have recognized the need for a fluid collection/delivery device that determines and/or collects information associated with the delivery to and/or receipt of fluid from a subject.

The type of collected information associated with the delivery to and/or receipt of fluid from a subject includes, and is not limited to, the date and/or time that fluid or other substance is delivered to and/or received from a subject, analytical properties associated with a sample that is received from a subject, such as pH level of the sample, temperature of the sample, hemoglobin concentration, oxygen levels, viscosity, and so on. As used herein, the words “collect(s) information” or “collecting information” do not require a permanent storage or record of such information—rather, the information may be temporarily stored.

Fluids received from the skin and/or from beneath the skin of the subject will often contain various analytes within the body that are important for diagnostic purposes, for example, markers for various disease states, such as glucose (e.g., for diabetics); other example analytes include ions such as sodium, potassium, chloride, calcium, magnesium, and/or bicarbonate (e.g., to determine dehydration); gases such as carbon dioxide or oxygen; H⁺ (i.e., pH); metabolites such as urea, blood urea nitrogen or creatinine; hormones such as estradiol, estrone, progesterone, progestin, testosterone, androstenedione, etc. (e.g., to determine pregnancy, illicit drug use, or the like); or cholesterol. Other examples include insulin, or hormone levels. Still other analytes include, but not limited to, high-density lipoprotein (“HDL”), low-density lipoprotein (“LDL”), albumin, alanine transaminase (“ALT”), aspartate transaminase (“AST”), alkaline phosphatase (“ALP”), bilirubin, lactate dehydrogenase, etc. (e.g., for liver function tests); luteinizing hormone or beta-human chorionic gonadotrophin (hCG) (e.g., for fertility tests); prothrombin (e.g., for coagulation tests); troponin, BNT or B-type natriuretic peptide, etc., (e.g., as cardiac markers); infectious disease markers for the flu, respiratory syncytial virus or RSV, etc.; or the like.

The inventors have appreciated that it is difficult to sterilize medical devices having electronic components. Some heat-based sterilization processes require high temperatures or steam that may damage electronic components. Some chemical methods may involve flammable and/or carcinogenic materials, and thus cannot be used to sterilize medical device. Other chemical methods may be used to sterilize electronics, but in some embodiments, where a medical device is stored in a sealed pouch, chemicals may be unable to access the device, and thus such chemical sterilization methods may not be effective. Some sterilization processes require application of vacuum which may damage electronic components. Some sterilization methods use radiation that may damage semiconductors. According to one aspect of the invention, a medical device contains two portions: an information collecting portion and a fluid receiving portion. In some embodiments, the information collecting portion and the fluid receiving portion can each stand alone as individual devices (i.e., an information collecting device and a fluid receiving device.) The information collecting portion may have an actuator and the fluid receiving device may have its own actuator, separate from the actuator of the information collecting portion. Each actuator may operate manually, mechanically, electrically, pneumatically, electromagnetically, or other suitable mode of operation, and may or may not require user input for activation. In some embodiments, the information collecting portion contains electronic components. As such, the fluid receiving portion may be sterilized while physically separated from the information collecting portion. In such embodiments, the information collecting portion does not receive fluid from a subject, deliver substances to a subject or otherwise invasively interact with a subject in a manner that would require sterilization, and thus the information collecting portion need not be sterilized. In some embodiments, the only electronic components of the device are located within the information collecting portion. The inventors have appreciated that gamma sterilization may be used to sterilize gamma stable electronic components. However, some electronic components, such as certain types of memory, are not gamma stable and thus cannot be subjected to gamma sterilization. Thus, in some embodiments, all gamma unstable electronic components are located within the information collecting portion. Within these embodiments, in some cases, gamma stable electronic components may be located within the fluid receiving device. In some embodiments, the information collecting portion is constructed and designed to attach to the fluid receiving portion. In some embodiments, actuation of the information collecting portion automatically actuates the fluid receiving portion as well.

The inventors have appreciated that a compact design is desirable for ease of transport, storage, and operation. The inventors have recognized that low-profile designs may enable one-hand operation, which can improve ease of use. According to one aspect of the invention, the information collecting portion has a compact, low-profile design.

The inventors have created a relative time keeping method that may help to lower power consumption and increase accuracy. The inventors have appreciated that lowering power consumption may increase shelf life, stability and allow for a more compact design. According to one aspect, a relative time keeping method is used to determine the time and date at which an event associated with a fluid receiving portion occurs. Of course, it should be appreciated that other time keeping methods are possible as well.

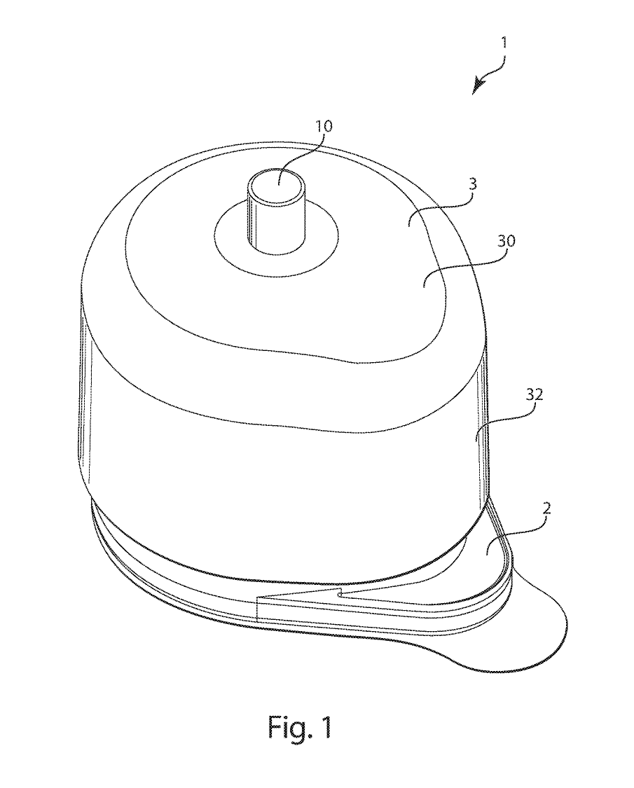

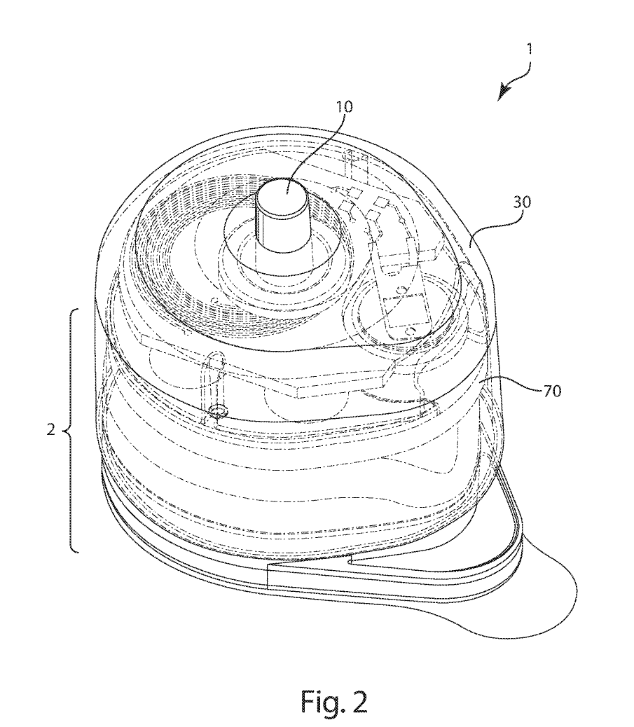

Turning now to the figures, FIG. 1 shows a device 1 that incorporates various aspects of the invention. Although FIG. 1 incorporates many of the aspects of the invention, any suitable number of aspects of the invention may be incorporated into a device. Thus, aspects of the invention may be used alone or in any suitable combination with each other. This illustrative embodiment includes a fluid receiving portion 2 and an information collecting portion 3. While the term “fluid receiving portion” or “fluid receiving device” will be used throughout the application, it should be appreciated that, as an alternative or as an addition to receiving fluid from a subject, this portion/device may be used to deliver substances to the subject. In some embodiments, as shown in FIG. 1, the information collecting portion 3 has an actuator 10 and a housing including a cover 30 and a bottom support 70 (see FIGS. 2, 4 and 5). The housing may be formed from or otherwise include Polyester (PCTA or PETG) or other polymers with low gas permeability. In some embodiments, the cover 30 extends downwardly over the fluid receiving portion 2 to surround at least a portion of the fluid receiving portion 2. FIG. 2 shows the device 1 with the cover 30 shown in phantom to reveal the components of the information collecting portion 3 and to show the fluid receiving portion 2 beneath. The components of the information collecting portion 3 will be discussed in detail in a later section. Although the device actuator 10 in this embodiment is arranged to be actuated by a user (e.g., by the press of a finger), the device actuator 10 may be arranged in other ways, e.g., for actuation by a machine, an electrical signal, or other suitable arrangement to cause the information collecting portion to collect information and/or to cause the fluid receiving portion 2 to receive fluid from a subject. Actuation of the device actuator 10 may occur automatically, e.g., in response to an elapsed timer or other stimulus or condition, or manually. In some embodiments, the device actuator 10 may include a push-button as shown, a sliding button, a touch-screen interface, a switch, or other user-actuatable arrangement, etc. In some cases, the device actuator 10 may allow for actuation of the device 1 only once, e.g., the device actuator 10 may become locked in a position that prevents further actuation, or may allow the device 1 to be actuated multiple times. In some embodiments, the information collecting portion is reusable such that it can be used with different fluid receiving devices. For example, the information collecting portion may be used with a first fluid receiving device, during which the device actuator 10 is actuated. Next, the information collecting portion may be used with a different, second fluid receiving device, during which the device actuator 10 is actuated again. The information collecting portion can be used with fluid receiving devices of different sizes and shapes, and is not limited for use with the information collecting portion shown in the figures.

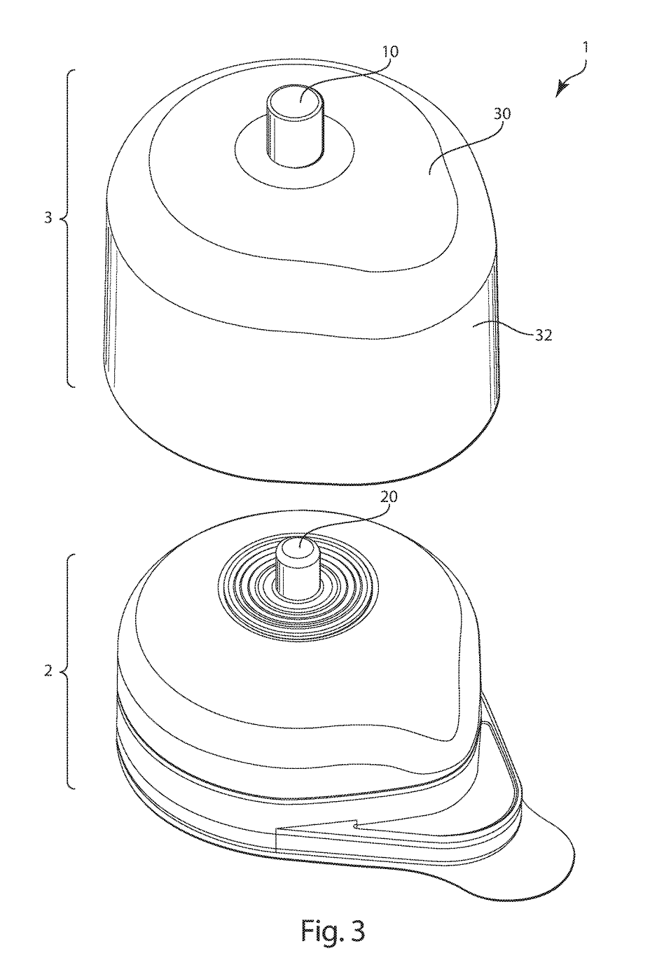

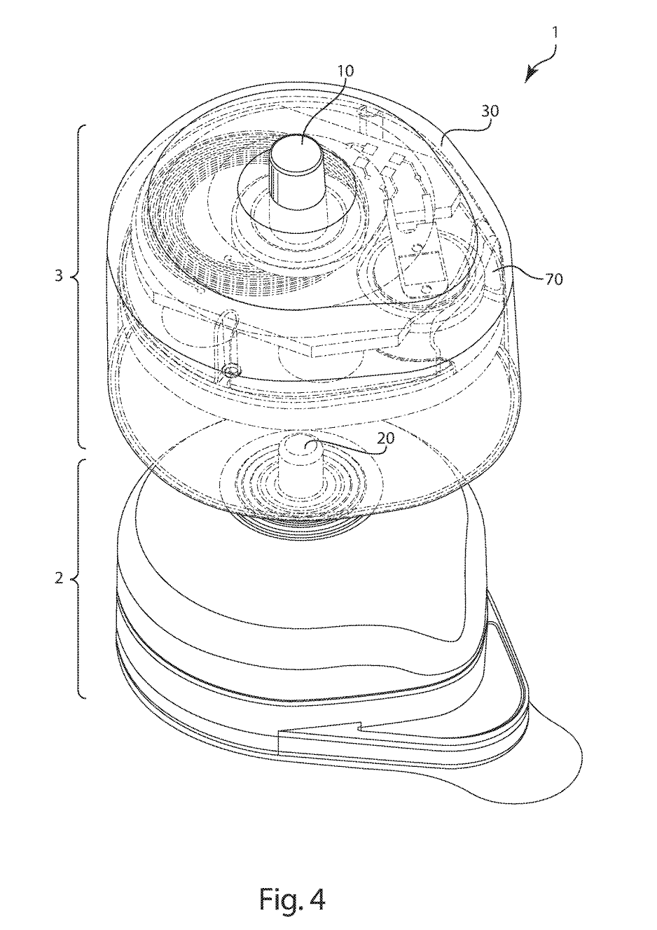

As mentioned above, according to one aspect, the device contains two portions: an information collecting portion and a fluid receiving portion. In some embodiments, the two portions are constructed and designed to be physically separable from one another. FIGS. 3-4 depict one embodiment of the device, where the information collecting portion 3 has been physically separated from the fluid receiving portion 2. In some embodiments, the fluid receiving portion 2 has its own actuator 20 that is separate from the actuator 10 of the information collecting portion 3. In some embodiments, the fluid receiving portion 2 may be operated as a stand-alone device, independent of the information collecting portion. In situations where no information collection is needed, a user may operate just the fluid receiving portion 2 by activating actuator 20 without needing the information collecting portion 3. In such situations, the information collecting portion 3 may be detached from the fluid receiving portion 2, or the information collecting portion 3 may be attached to the fluid receiving portion 2 and simply left unactuated.

In some embodiments, the fluid receiving portion and the information collecting portion may be constructed and designed to attach together. In some embodiments, the fluid receiving portion and the information collecting portion may be constructed and designed to attach together for operation of both portions with one another. In some embodiments, after the fluid receiving portion and the information collecting portion have been attached together, the two portions are permanently attached together such that they cannot be removed from one another. In other embodiments, the two portions are removeably attached together such that any user can detach the fluid receiving portion from the information collecting portion. In yet other embodiments, after the fluid receiving portion and the information collecting portion have been attached together, the two portions may be limitedly detachable from one another, such that some requirement (e.g., for safety, security, accuracy and/or quality assurance purposes) must be met before the portions can be detached from one another. For example, a specialized unlocking tool, passcode, finger/retina scan, environment detection (e.g., the portions unlock once a sensor detects that the portions have entered the analysis lab or machine) or other suitable requirement is needed to detach the portions from one another.

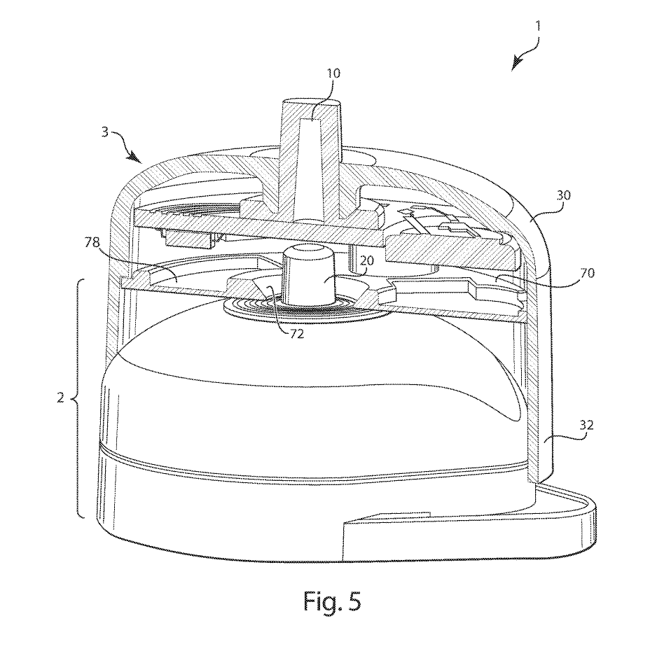

In the embodiment shown in FIGS. 3-5, the information collecting portion 3 and the fluid receiving portion 2 are constructed and designed to attach together by having the information collecting portion 3 cover a top portion of the fluid receiving portion 2. In some embodiments, as seen in FIG. 5, the information collecting portion 3 includes a cavity into which the fluid receiving portion 2 is received. In some embodiments, the information collecting portion 3 receives at least a portion of the actuator 20 of the fluid receiving portion 2. In some embodiments, the actuator 20 is fully received within the information collecting portion 3. In some embodiments, the information collecting portion 3 attaches to the fluid receiving portion 2 via a snap-fit type engagement. As shown in FIGS. 2 and 5, the cover 30 of the information collecting portion 3 has a downwardly extending sidewall 32 that extends over the internal components of the information collecting portion 3 and has a long enough height to also extend over at least a portion of the fluid receiving portion 2. The cover 30 may surround at least a portion of the fluid receiving portion 2. The fluid receiving portion 2 may attach to the information collecting portion 3 by an interference fit, mechanical interlock, hook and loop type fasteners, threaded connection, mechanical fasteners, pressure sensitive adhesives, magnets, or by any other suitable arrangement. The two portions may be constructed and designed to attach to one another via a third intermediate component such as a coupling, or may be directly attached to one another. In some embodiments, the information collecting portion 3 may attach to the fluid receiving portion by stacking the information collecting portion 3 on top of the fluid receiving portion, and having at least a portion of the fluid receiving portion received within a cavity of the information collecting portion. Alternatively, at least a portion of the information collecting portion may be received within the fluid receiving portion. In some embodiments, the fluid receiving portion may attach to the information collecting portion by stacking the fluid receiving portion on top of the information collecting portion.

It should be appreciated that, in other embodiments, attaching the fluid receiving portion to the information collecting portion is not necessary. For example, the two portions may not be attached together, but commands and/or information may be transmitted wirelessly between the two portions.

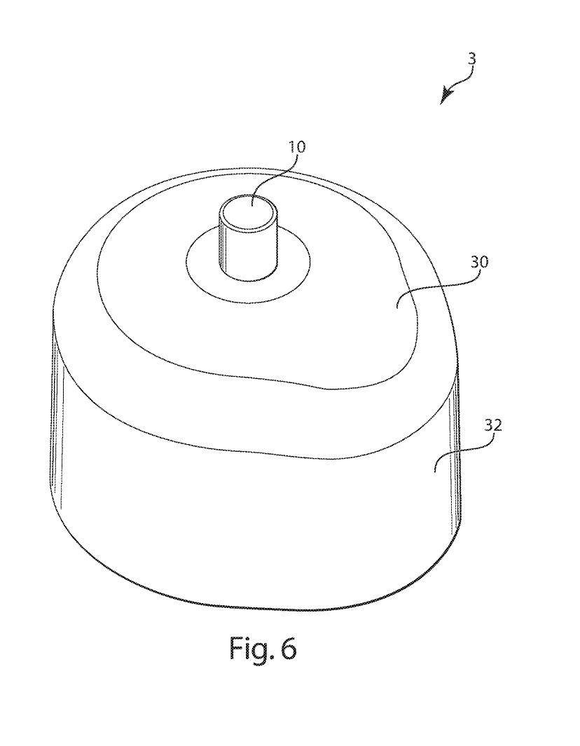

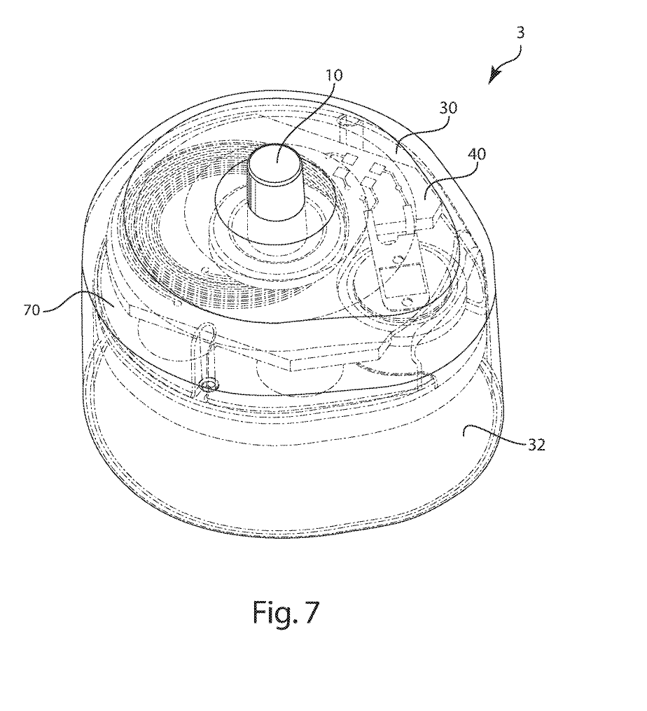

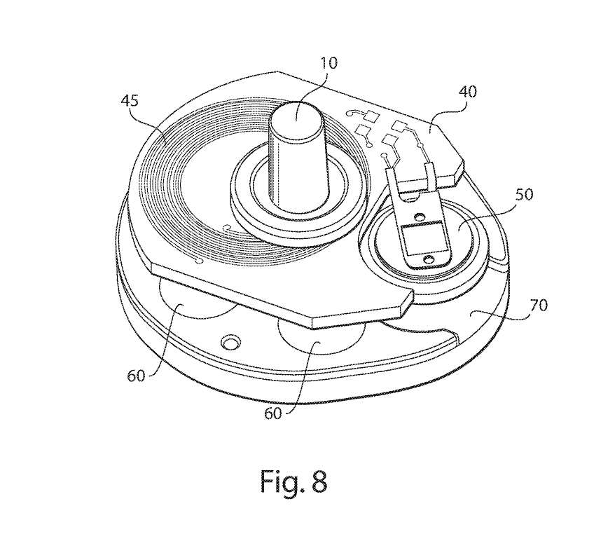

As discussed above, in some embodiments, actuation of the information collecting portion automatically actuates the fluid receiving portion as well. According to one embodiment, as seen in FIG. 6, the information collecting portion 3 includes an actuator 10 and a housing including a cover 30, the cover 30 having a sidewall 32. In the embodiment shown in FIG. 6, the actuator 10 is pressed downwardly by a user and is moveable relative to the housing. As seen in FIG. 7, where the cover 30 is shown in phantom to reveal the inner components of the information collecting portion, and as seen in FIG. 8, where the cover 30 is removed altogether, the actuator 10 is attached to a circuit board 40. In some embodiments, the circuit board is a printed circuit board. In some embodiments, the actuator 10 is rigidly fixed to the circuit board 40 such that movement of the actuator 10 causes the circuit board 40 to move with the actuator 10. As such, the circuit board 40 is also moveable relative to the housing (e.g. cover 30 and bottom support 70) of the information collecting portion.

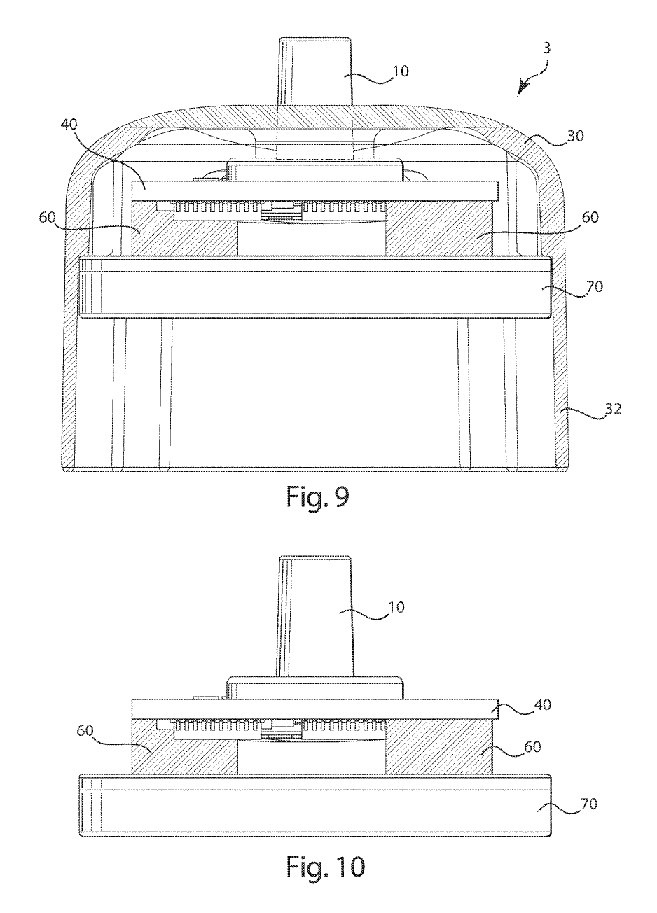

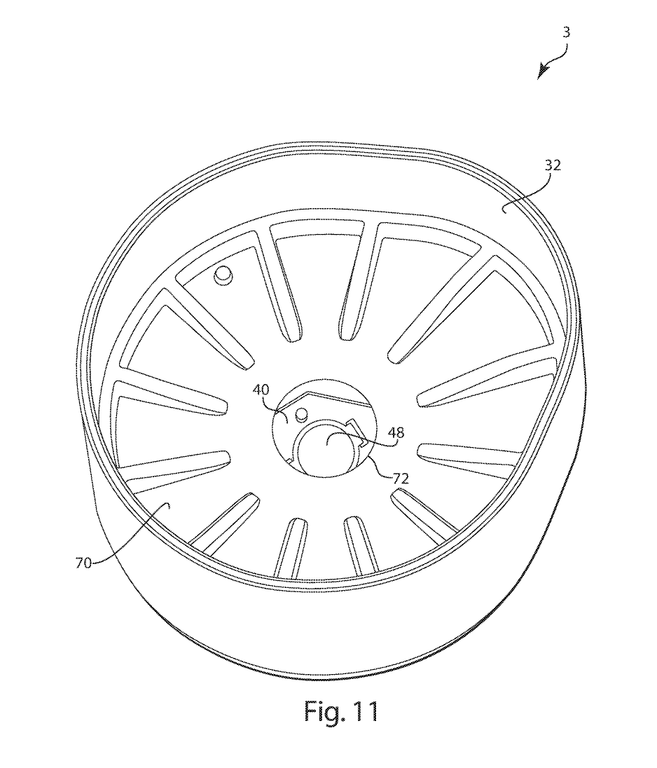

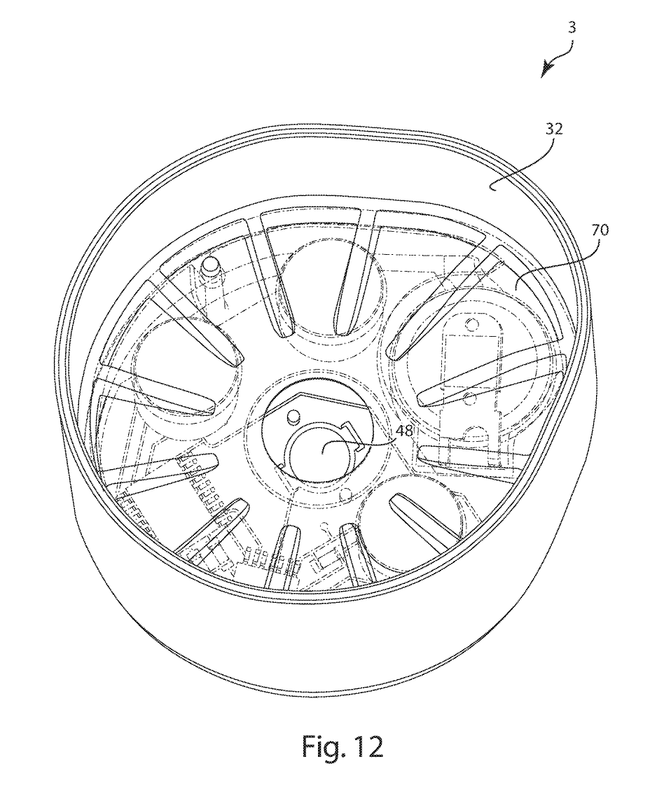

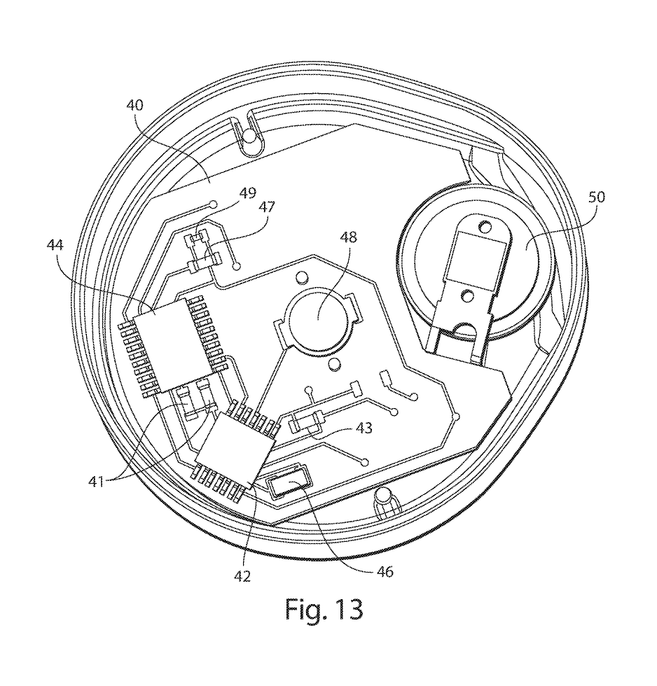

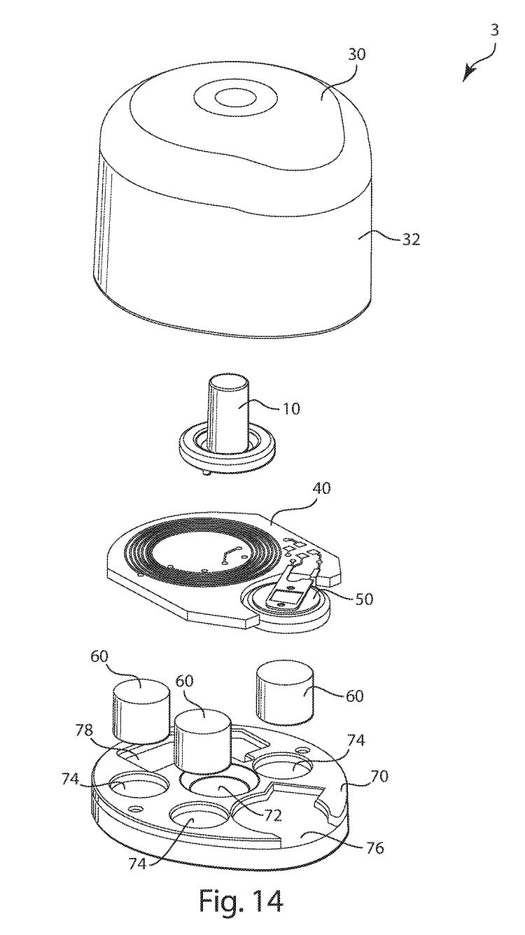

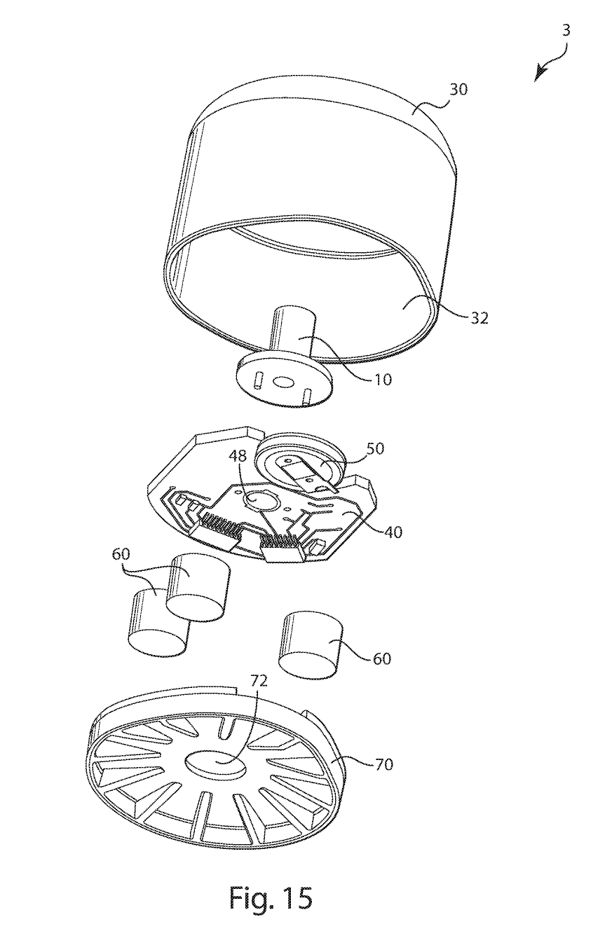

In accordance with one embodiment seen in FIG. 9, which is a side view of the information collecting portion with the cover 30 shown in phantom, the actuator 10 extends through a top opening in the cover 30 such that a user can access the top of the actuator 10. The circuit board 40 is supported in a pre-actuation position by spacers 60. The spacers 60 are supported by a bottom support 70 which is fixed to the cover 30. In some embodiments, the spacers 60 are compressible to permit movement of the circuit board 40 and actuator 10 relative to the housing (e.g., cover 30 and bottom support 70). As more clearly seen in FIG. 10, in which the cover 30 is removed, the spacers 60 rest upon the bottom support 70 and support the circuit board 40. As seen in FIG. 11, the bottom support includes a through-hole 72, which provides access to a switch 48 located on the underside of the circuit board 40. As seen in FIG. 12, where the bottom support 70 is shown in phantom, the underside of the circuit board 40 includes various components that will be described in detail in a later section. As seen in FIG. 13, in which the bottom support 70 is removed, the switch 48 is connected to a circuit mounted to the circuit board 40. In some embodiments, actuation of the switch 48 causes the information collecting portion 3 to begin a process of collecting and/or determining information. The through-hole 72 of the bottom support 70 and the switch 48 on the underside of the circuit board 40 can be seen in exploded views FIGS. 14-15. In some embodiments, the switch 48 is aligned with the through-hole 72 such that an object entering through the hole 72 may actuate the switch.

In some embodiments, the spacers 60 are made of a compressible material or structure such that the spacer is compressible to a shorter height when subjected to a compression force, and then, when the compression force is removed, the spacer regains its original height. The spacers may be made of foam, springs (helical, helical cone, leaf, volute, etc.), Belleville washers, or any other suitable material and/or structural arrangement.

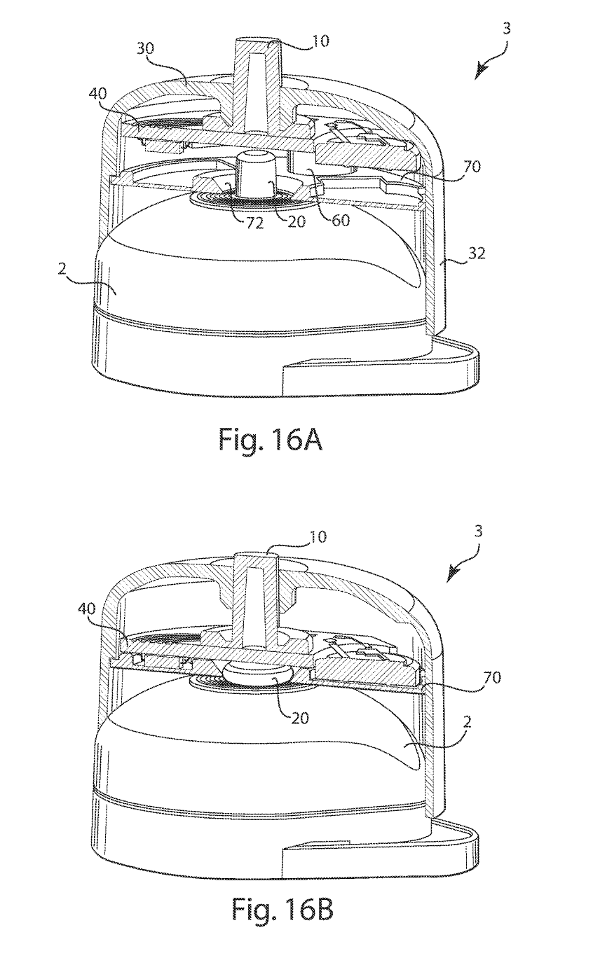

The actual actuation process is best seen in FIGS. 16A-16B. As seen in FIG. 16A, the information collecting portion 3 is attached to the fluid receiving portion 2. The sidewall 32 of the cover 30 of the information collecting portion 3 extends over a portion of the fluid receiving portion 2. In addition, the actuator 20 of the fluid receiving portion 2 extends partially into the information collecting portion 3 through the through-hole 72 of the bottom support 70. In the pre-actuation state, seen in FIG. 16A, the underside of the circuit board 40 is spaced from the actuator 20 of the fluid receiving portion 2. The spacer 60 supports the circuit board 40 in this elevated, pre-actuation position. In some embodiments, activation of the device actuator causes the information collecting portion to transmit a force to the fluid receiving portion. In turn, the force actuates the fluid receiving portion to begin a fluid collecting process. As seen in FIG. 16B, a user actuates the information collecting portion by pressing down on actuator 10, which in turn causes the circuit board 40 to move downward. The spacer 60 is compressible, and permits circuit board 40 to move downward due to the application of force on the actuator 10. As the circuit board 40 moves downward toward the fluid receiving portion 2, the underside of the circuit board 40 actuates the fluid receiving portion 2 by contacting and activating the actuator 20 of the fluid receiving portion 2. As such, the information collecting portion 3 transmits force that is applied to the actuator 10 to the fluid receiving portion actuator 20. As discussed previously, a switch 48 on the underside of the circuit board 40 (seen in FIG. 15) is aligned with the through-hole 72. Contact between the top of the actuator 20 with the switch 48 actuates the switch 48 and triggers the information collecting portion 3 to begin collecting information. For example, if the information collecting portion 3 collects information associated with the time and date at which the fluid receiving portion is actuated, contact between the top of the actuator 20 with the switch 48 initiates a process for collecting such information.

The switch may be a momentary switch, a latching switch, or any other suitable type of switch. In some embodiments, the switch comprises a snap dome, where tripping of the snap dome closes a circuit. For example, in one embodiment, the circuit includes two contacts. The snap dome feet or legs rest upon or are soldered to the first contact. Tripping of the snap dome (e.g., deflection of the snap dome to a concave up configuration) causes the center of the snap dome to contact the second contact. Contact of the snap dome with the second contact closes the circuit. In some cases, the snap dome is monostable, meaning that the snap dome is biased to return to the same configuration after applied force is removed from the snap dome. With a monostable snap dome, an applied force may trip the snap dome and move it from a first configuration to a second configuration (e.g., concave up to concave down), but upon removal of the applied force, the snap dome automatically snaps back to its first configuration. In other embodiments, the snap dome is bistable, meaning that, when a force is applied to trip the snap dome and move it from a first configuration to a second configuration, the snap dome remains in the second configuration even after removal of the applied force.

The snap dome may be of any suitable shape and/or size. For example, the snap dome may be circular (having no “legs”), oblong, triangular (have 3 legs), square (4 legs with straight sides between each leg), pentagonal (5 legs), hexagonal (6 legs), spider-legged, star-like, clover-shaped (with any number of lobes, e.g., 2, 3, 4, 5, etc.), a serrated disc or a wave shape, or the like. The snap dome may be formed from or otherwise include any suitable material, for example, a metal such as stainless steel (e.g., 301, 301LN, 304, 304L, 304LN, 304H, 305, 312, 321, 321H, 316, 316L, 316LN, 316Ti, 317L, 409, 410, 430, 440A, 440B, 440C, 440F, 904L), carbon steel, spring steel, spring brass, phosphor bronze, beryllium copper, titanium, titanium alloy steels, chrome vanadium, nickel alloy steels (e.g., Monel 400, Monel K 500, Inconel 600, Inconel 718, Inconel x 750, etc.), or the like.

In some embodiments, all components of the circuit are surface mounted to the circuit board, except for the battery. In some embodiments, the battery is surface-mounted as well. In other embodiments, some or all components are through-hole soldered to the circuit board rather than surface-mounted.

The time-keeping aspect of the information collecting portion will now be discussed. In some embodiments, the information collecting portion may have a switch or triggering component that, when tripped or triggered, indicates that some event of the fluid receiving portion and/or the information collecting portion has occurred. Non-limiting, illustrative examples of possible events include: delivery of a substance to a subject, receipt of fluid from a subject, piercing of a subject by one or more needles or microneedles of the fluid receiving portion, an in situ analytical test has been completed (e.g., in an analytical chamber of the fluid receiving portion or the information collecting portion), a threshold level has been exceeded (e.g. concentration of an analyte or drug), and so on. In some embodiments, the information collecting portion includes a monitoring circuit. In some cases, a circuit may repeatedly sample the condition of the circuit. Tripping of the switch may cause the circuit to open, which communicates to a controller such as a microcontroller or microprocessor that the switch has been tripped and, accordingly, an event has occurred. A battery may be included to power the circuit. In other embodiments, tripping of the switch closes a circuit, which communicates to a controller that the switch has been tripped and, accordingly, an event has occurred. As will be discussed in more detail below, in relative time-keeping mode, when the switch has been tripped, the controller begins counting time (e.g., like a stopwatch counting upward) using a crystal oscillator. In absolute time-keeping mode, the controller, which has been counting time as soon as the circuit is assembled and the controller is programmed, stops counting when the switch is tripped and/or marks the time at which the switch has been tripped. The controller may then communicate such information to another device by radio frequency (e.g., by using an RFID transponder and antenna), free-space optical communication, electromagnetic induction, a wired connection, or by any other suitable arrangement.

In some embodiments, the information collecting device may be physically sent to a lab or other data processing center. The lab or data processing center may receive the time-keeping information from the information collecting device, as well as any other data or information associated with a stored sample or with operation of the delivery portion/fluid receiving portion. In some cases, the information collecting device is sent along with the sample contained within the fluid receiving portion. The sample may undergo further analysis that may be correlated from the information from the information collecting portion.

In one illustrative embodiment, shown in FIGS. 6-15, the information collecting portion includes a circuit board with several components. The circuit board has a first side and a second side. In some embodiments, the circuit board is double sided such that both sides of the circuit board support components. As best seen in FIG. 8, the information collecting portion actuator 10 is fixed to the first side of the circuit board. The actuator 10 may be attached to the circuit board 40 by adhesive (e.g. tape, liquid), mechanical fastening (e.g. interference fit, slot/groove, screws) or thermal methods (e.g. heat staking, welding, soldering), or otherwise attached to the circuit board by any suitable arrangement. The circuit board also includes an antenna 45 and a battery 50. On the underside of the circuit board 40, best seen in FIG. 13, the circuit board may include a switch 48, microcontroller 42, transponder 44, crystal oscillator 46, resistors 41 and 43, Zener diode 47 and capacitor 49. Turning to FIGS. 14-15, as discussed previously, a bottom support 70, which is fixed to the cover, supports one or more spacers 60 that hold the circuit board 40 up such that the underside of the circuit board is spaced from the bottom support. The spacers 60 may be attached to the bottom support, for example, using adhesive. The top side of the bottom support may include depressions that receive the spacers 60 and/or the components on the underside of the circuit board 40 when the spacers 60 are compressed and the circuit board 40 is moved downward toward the bottom support 70. For example, as seen in FIG. 14, depressions 74 in the bottom support 70 are shaped and positioned to receive the spacers 60. Depression 76 is shaped and positioned to receive the battery 50 and depression 78 is shaped and positioned to receive other components such as the microcontroller 42 and transponder 44.

In some embodiments, the switch is a NICOMATIC SMT dome reference 170 having a trip force of 170±20 grams, a height of 0.30±0.08 mm and a total travel of 0.20±0.08 mm. In some embodiments, the microcontroller 42 is TEXAS INSTRUMENTS MSP430F2012. In some embodiments, the transponder 44 is MELEXIS MLX90129. In some embodiments, the crystal oscillator 46 is TXC CORPORATION 9HT10-32.768KDZF-T, which keeps time to <20 ppm. In some embodiments, the battery is PANASONIC BSG BR-1225A/FAN, which is a 3V lithium single cell battery. In some embodiments, the resistors 41 are 10 KΩ resistors and the resistor 43 is a 47 KΩ resistor. It should be appreciated that other components may be used, as the invention is not limited to these specific components.

Time keeping processes will now be discussed. First, the relative time keeping method will be discussed. According to some embodiments, actuation of the actuator 10 trips the switch 48, as discussed previously. In the relative time keeping method, when the switch is tripped, the microcontroller 42 begins counting upward in time, using the crystal oscillator 46 to maintain an accurate count. The time count may be stored in a memory of the microcontroller 42 or may be transferred to a memory on the transponder 44. The microcontroller 42 sends the time count information to the transponder 44 either periodically or only when requested by a reader. In some embodiments, the transponder 44 is an RFID device with onboard user memory and an ability to communicate with the microcontroller 42. The antenna 45 transmits the memory contents of the transponder and/or memory contents of the microcontroller 42 that contains information including the time count information from the transponder 44 to a reader. The reader may be external to the information collecting portion. The information that is transmitted from the transponder 44 to the reader contains information used to determine the time that has elapsed since actuation of the actuator 10. The reader and/or a computer or other processing device determines the exact time at which the information is read from the transponder. A computer or other processing device subtracts the elapsed time from the time that the information is read to arrive at the time and date at which the actuator 10 was originally actuated.

In some embodiments, the microcontroller 42 and the transponder 44 are two separate integrated circuits. In other embodiments, the transponder functionalities are integrated on the same integrated circuit as the microcontroller 42. In some embodiments, the time keeping functions are implemented in the microcontroller 42, while in other embodiments, the time keeping functions are carried out by a separate integrated circuit that functions as a clock or a real-time clock. In some embodiments, the time keeping functions are carried out on the same integrated circuit as the transponder 44, which may be on the same integrated circuit as the microprocessor 42.

It should be appreciated that alternative ways of transmitting information may be used. For example, information transmission may be accomplished using a visual display, an audio output, a physical wired connection, wireless modalities (e.g., Bluetooth, low energy Bluetooth, infrared, etc.) In alternative embodiments, the transponder 44 may be an infrared data transmitter with an ability to communicate with the microcontroller 42, a radio frequency (RF) data transmitter with an ability to communicate with the microcontroller 42 utilizing a commercially available protocol such as Bluetooth, Bluetooth low energy, ZigBee, Z-wave or any other such protocols, or a radio frequency (RF) data transmitter with the means to communicate with the microcontroller 42 utilizing a proprietary communication protocol.

In the absolute time keeping method, the information collecting portion is set to a universal clock and begins counting as soon as the circuit is assembled and the microcontroller is programmed. When the switch is tripped, the microcontroller stops counting and/or marks the time at which the switch was tripped. The information that is transmitted from the transponder to a reader is the absolute time at which the actuator 10 was actuated.

In some embodiments, the information collecting device may be physically sent to a lab or other data processing center. The lab or data processing center has the reader that reads the information from the transponder. In some cases, the information collecting device is sent along with the sample contained within the fluid receiving portion. The sample may undergo further analysis that may be correlated from the information from the transponder.

The fluid receiving portion will now be discussed. As discussed above, while the portion/device has been referred to as a “fluid receiving portion,” it should be appreciated that this portion/device may be used to deliver substances to the subject alternatively or in addition to receiving fluid from the subject.

In one illustrative embodiment, the fluid receiving portion 2 may include an opening through which fluids from the body may be received. The opening may have any suitable shape. For example, the opening can be generally hemispherical, semi-oval, rectangular, irregular, etc. The opening may lie in a two-dimensional plane or the opening may include a three-dimensional cavity, hole, groove, slit, etc. In some embodiments, the fluid receiving portion may include a flow activator, such as one or more needles or microneedles, arranged to cause fluid to be released from the subject, e.g., by piercing the skin of a subject. It should be noted that a flow activator need not be included with all embodiments as the device may not necessarily employ a mechanism for causing fluid release from the subject. For instance, the device may receive fluid that has already been released due to another cause, such as a cut or an abrasion, fluid release due to a separate and independent device, such as a separate lancet, an open fluid access such as during a surgical operation, and so on. Additionally, fluid may be introduced into the device via urination, spitting, pouring fluid into the device, etc. If included, a flow activator may physically penetrate, pierce, and/or or abrade, chemically peel, corrode and/or irritate, release and/or produce electromagnetic, acoustic or other waves, or otherwise operate to cause fluid release from a subject. The flow activator may include a moveable mechanism, e.g., to move a needle, or may not require movement to function. For example, the flow activator may include a jet injector or a “hypospray” that delivers fluid under pressure to a subject, a pneumatic system that delivers and/or receives fluid, a hygroscopic agent that adsorbs or absorbs fluid, a reverse iontophoresis system, a transducer that emits ultrasonic waves, or thermal, radiofrequency and/or laser energy, and so on, any of which need not necessarily require movement of a flow activator to cause fluid release from a subject.

In one illustrative embodiment, the flow activator of the fluid receiving portion 2 includes one or more needles or microneedles that are moveable relative to the housing of the fluid receiving portion 2 such that the needles or microneedles are used to pierce the skin of a subject.

The fluid receiving portion 2 may include a storage chamber in which fluids that are received by the fluid receiving portion are stored. A channel may fluidly connect the opening to the storage chamber. In some embodiments, on-board, pre-packed vacuum is stored within the fluid receiving portion 2 during production and assembly of the fluid receiving portion (i.e. such that vacuum exists within the fluid receiving portion prior to actuation of the fluid receiving portion). In other words, a volume of space in the fluid receiving portion is evacuated to create vacuum inside the volume of space during production and assembly of the fluid receiving portion. Such pre-packed vacuum may facilitate movement of fluid from the opening into the storage chamber.

In some embodiments, the fluid receiving portion receives fluid but does not deliver substances to a subject. The fluid receiving portion may contain the on-board, pre-packed vacuum discussed above. In some embodiments, the fluid receiving portion does not pierce skin or otherwise initiate fluid release from a subject (e.g., the fluid receiving portion has no needles), but the on-board pre-packed vacuum facilitates collection of fluid into the fluid receiving portion. For example, in one embodiment, the fluid receiving portion is an evacuated vacuum tube, such as, but not limited to, the VACUTAINER (Becton, Dickinson and Company) or the VACUETTE (Greiner Bio-One GMBH).

In some embodiments, the fluid receiving portion is actuated to begin a fluid collecting process by an action other than a button press. For example, with some evacuated vacuum tubes such as a VACUTAINER, the evacuated vacuum tube begins a fluid collecting process when a user inserts a blood collection needle through a seal on the tube to access the vacuum. In some embodiments, a user may actuate the fluid receiving portion to begin a fluid collecting process, and around the same time, simultaneously, or some time before or afterwards, actuate the information collecting portion to collect information. The actuator of the information collecting portion and the actuator of the fluid receiving portion may be distinct components. In some embodiments, a sensor may detect when the fluid receiving portion has been actuated to begin a fluid collecting process. For example, the sensor may detect a pressure change within the fluid receiving portion, entry of fluid, change in pH, change in temperature, change in humidity, or any other suitable characteristic. The sensor may communicate to the information collecting portion that the fluid receiving portion has been actuated and may cause the information collecting portion to begin collecting information.

In one embodiment, the information collecting portion includes a needle that is constructed and designed to actuate an evacuated vacuum tube to being a fluid collecting process.

In one embodiment, an actuator button is located on an end of an evacuated vacuum tube. When a user moves the tube toward a blood collection needle, the user simultaneously presses on the actuator button, which actuates the information collecting portion to begin an information collecting process. To prevent inadvertent actuation of the actuator button, the actuator button may be recessed and/or have a removable covering.

In some embodiments, the information collecting portion may be used in conjunction with a flow activator device such as a lancet or needle that causes fluid release from a subject, but does not collect fluid. The information collecting portion may collect information relating to the date and time of actuation of the flow activator. Similar to the discussion above with the fluid receiving portion, the information collecting portion may be constructed and designed to attach to the flow activator device. After the information collecting portion is attached to the flow activator device, the information collecting portion may be detachable from the flow activator device, permanently attached to the flow activator device, or limitedly detachable from the flow activator device.

The fluid receiving portion 2 and/or the information collecting portion 3 may include an anticoagulant or a stabilizing agent for stabilizing the fluid withdrawn from the skin and/or beneath the skin. As a specific non-limiting example, an anticoagulant may be used for blood withdrawn from the skin. Examples of anticoagulants include, but are not limited to, heparin, citrate, thrombin, oxalate, ethylenediaminetetraacetic acid (EDTA), sodium polyanethol sulfonate, acid citrate dextrose. Other agents may be used in conjunction with or instead of anticoagulants, for example, stabilizing agents such as solvents, diluents, buffers, chelating agents, enzyme inhibitors (i.e., protease or nuclease inhibitor), antioxidants, binding agents, preservatives, antimicrobials, or the like. Examples of preservatives include, for example, benzalkonium chloride, chlorobutanol, parabens, or thimerosal. Non-limiting examples of antioxidants include ascorbic acid, glutathione, lipoic acid, uric acid, carotenes, alpha-tocopherol, ubiquinol, or enzymes such as catalase, superoxide dismutase, or peroxidases. Examples of microbials include, but are not limited to, ethanol or isopropyl alcohol, azides, or the like. Examples of chelating agents include, but are not limited to, ethylene glycol tetraacetic acid or ethylenediaminetetraacetic acid. Examples of buffers include phosphate buffers such as those known to ordinary skill in the art.

The fluid receiving portion of the device and/or the information collecting portion of the device may include one or more sensors for detecting one or more characteristics of a fluid received from a subject. The sensors may detect characteristics of the fluid at any point in time along the handling process of the fluid, e.g., upon entry into the fluid receiving device, upon storage within the fluid receiving device, as the fluid receiving device is prepped for shipping with the fluid stored held within the fluid receiving device, during the transportation of the fluid receiving device, when the fluid receiving device arrives at its final destination, or any other suitable point in time. Monitoring characteristics of the fluid at these different time points may be useful in determining whether the fluid was subjected to certain environmental changes that may have changed the characteristics of the fluid. For example, sensors can monitor the temperature, pH, oxygen levels, or any other suitable characteristic of the fluid within the fluid receiving portion as it is transported.

Other types of information may be monitored and recorded/transmitted by the information collecting portion as well. For example, information may include the time at which fluid enters the fluid receiving portion, the time at which the fluid receiving portion becomes full, coordinates of the fluid receiving portion, tracking information (e.g. similar to tracking mail and packages), the time at which the fluid receiving portion was shipped and/or delivered to its final destination and the time at which the fluid sample was put on ice, refrigerated or otherwise cooled. The information may include characteristics of the fluid receiving portion itself, for example, the stability, oxygen content, or other characteristics, which may be measured at different time points along the handling process of the fluid receiving portion, e.g., as soon as the fluid receiving portion is manufactured, just prior to use for receiving a fluid, as the fluid receiving portion is transported, and/or when the fluid receiving portion arrives at its final destination for analysis. In sum, any characteristic of the fluid or the fluid receiving portion may be monitored and collected by the information collecting portion as the fluid and/or the fluid receiving portion undergo a workflow process. The information collecting portion may collect a single piece of information or multiple pieces of information, may collect all data at once, at discrete intervals, or throughout a workflow process, and/or may track information at discrete intervals or continuously through time. The information collecting portion may also be re-useable. For example, the information collecting portion may be used in conjunction with a first fluid receiving portion, and afterwards, with a second fluid receiving portion. The information collecting portion may also be used with more than one fluid receiving portion simultaneously.

Sensor(s) may be located in any suitable way or location with respect to the device, such as in a storage chamber, in a channel, etc. For example, the device may include a pH sensor, an optical sensor, an oxygen sensor, a sensor able to detect the concentration of a substance, or the like. Non-limiting examples of sensors useful in the invention include dye-based detection systems, affinity-based detection systems, microfabricated gravimetric analyzers, CCD cameras, optical detectors, optical microscopy systems, electrical systems, thermocouples and thermistors, pressure sensors, etc. Those of ordinary skill in the art will be able to identify other suitable sensors. The sensor can include a colorimetric detection system in some cases, which may be external to the device, or microfabricated into the device in certain cases. As an example of a colorimetric detection system, if a dye or a fluorescent entity is used (e.g. in a particle), the colorimetric detection system may be able to detect a change or shift in the frequency and/or intensity of the dye or fluorescent entity. In some embodiments, or the sample from a subject may be analyzed within the device in situ, e.g., by adding one or more reaction entities to the device, for instance, to a storage chamber, or to analytical chamber within the device.

In one set of embodiments, the sensor may be a test strip, for example, test strips that can be obtained commercially. Examples of test strips include, but are not limited to, glucose test strips, urine test strips, pregnancy test strips, or the like. A test strip will typically include a band, piece, or strip of paper or other material and contain one or more regions able to determine an analyte, e.g., via binding of the analyte to a diagnostic agent or a reaction entity able to interact with and/or associate with the analyte. For example, the test strip may include various enzymes or antibodies, glucose oxidase and/or ferricyanide, or the like. The test strip may be able to determine, for example, glucose, cholesterol, creatinine, ketones, blood, protein, nitrite, pH, urobilinogen, bilirubin, leucocytes, luteinizing hormone, etc., depending on the type of test strip. The test strip may be used in any number of different ways. In some cases, a test strip may be obtained commercially and inserted into the device, e.g., before or after receiving blood, interstitial fluid, or other fluids from a subject. At least a portion of the blood or other fluid may be exposed to the test strip to determine an analyte, e.g., in embodiments where the device uses the test strip as a sensor so that the device itself determines the analyte. In some cases, the device may be sold with a test strip pre-loaded, or a user may need to insert a test strip in a device (and optionally, withdraw and replace the test strip between uses). In certain cases, the test strip may form an integral part of the device that is not removable by a user. In some embodiments, after exposure to the blood or other fluid withdrawn from the subject, the test strip may be removed from the device and determined externally, e.g., using other apparatuses able to determine the test strip, for example, commercially-available test strip readers.

While aspects of the invention have been described with reference to various illustrative embodiments, such aspects are not limited to the embodiments described. Thus, it is evident that many alternatives, modifications, and variations of the embodiments described will be apparent to those skilled in the art. Accordingly, embodiments as set forth herein are intended to be illustrative, not limiting. Various changes may be made without departing from the spirit of aspects of the invention.

What is claimed is:

1. A device for receiving fluid from a subject, comprising:
a device actuator;
a fluid receiving portion constructed and designed to receive fluid from the subject, the fluid receiving portion including a fluid storage chamber for storing the fluid released by the subject;
an information collecting portion being constructed and designed to attach to the fluid receiving portion, the information collecting portion being constructed and designed to collect information associated with operation of the fluid receiving portion or the fluid stored in the storage chamber,
wherein activation of the device actuator actuates the fluid receiving portion to begin a fluid collecting process and actuates the information collecting portion to collect information.

2. The device of claim 1, wherein:
the information collecting portion comprises a housing having sidewalls,
the fluid receiving portion comprises a housing, and
when the information collecting portion is attached to the fluid receiving portion, the sidewalls surround at least a portion of the housing of the fluid receiving portion.

3. The device of claim 2, wherein the housing of the information collecting portion attaches to the housing of the fluid receiving portion via a snap-fit engagement.

4. The device of claim 1, wherein all electronic components of the device are contained only within the information collecting portion.

5. The device of claim 1, wherein the fluid receiving portion comprises a fluid receiving device that is independently operable from the information collecting portion, which comprises an information collecting device.

6. The device of claim 1, wherein the information collecting portion further comprises a switch, wherein triggering of the switch actuates the information collecting portion to collect information.

7. The device of claim 6, wherein the fluid receiving portion further comprises an actuator.

8. The device of claim 7, wherein contact of the actuator of the fluid receiving portion with the switch triggers the switch and actuates the fluid receiving portion to cause release of fluid from a subject and receipt of fluid into the fluid receiving portion.

9. The device of claim 8, wherein activation of the device actuator causes the actuator of the fluid receiving portion to contact the switch.

10. The device of claim 8, further comprising a bottom support having a through-hole for receiving an actuator of a fluid receiving portion,
wherein the through-hole of the bottom support is aligned with the switch such that contact between the actuator of the fluid receiving portion and the switch triggers the switch and actuates the fluid receiving portion to cause release of fluid from a subject and receipt of fluid into the fluid receiving portion.

11. The device of claim 1, wherein the information collected by the information collecting device is associated with a time and date of actuation of the fluid receiving portion or receipt of fluid into the fluid receiving portion.

12. The device of claim 7, wherein the actuator of the fluid receiving portion is actuatable independently of the device actuator.

13. The device of claim 1, wherein the information collecting portion is removeably attachable to the fluid receiving portion.

14. The device of claim 1, wherein, after the information collecting portion is attached to the fluid receiving portion, the information collecting portion is permanently attached to the fluid receiving portion.

15. The device of claim 1, wherein, after the information collecting portion is attached to the fluid receiving portion, the information collecting portion is limitedly detachable from the fluid receiving portion.

16. The device of claim 1, wherein the fluid receiving portion is constructed and designed to cause fluid to be released from the subject, the fluid receiving portion including a needle for penetrating skin of a subject to cause release of fluid from the subject.

17. The device of claim 1, wherein the information collecting portion is reusable with a second fluid receiving portion.

18. The device of claim 1, wherein all gamma unstable electronic components of the device are contained only within the information collecting portion.

19. The device of claim 1, wherein activation of the device actuator causes the information collecting portion to transmit a force to the fluid receiving portion and the force actuates the fluid receiving portion to begin a fluid collecting process.

20. The device of claim 1, wherein the fluid receiving portion comprises an actuator and the information collecting portion receives at least a portion of the actuator of the fluid receiving portion.

21. The device of claim 1, wherein the information collecting portion covers a top portion of the fluid receiving device.

22. The device of claim 1, wherein the information collecting portion includes a cavity into which the fluid receiving portion is received.

23. An information collecting device for collecting information associated with release of fluid from a subject or receipt of fluid into a medical device, comprising:
a housing that is constructed and designed to attach to a fluid receiving device having a fluid storage chamber for storing the fluid released by the subject; and
a controller constructed and designed to collect information associated with operation of the fluid receiving device or fluid stored in the storage chamber of the fluid receiving device,
wherein the information collecting portion is actuated to collect information when the fluid receiving device undergoes a fluid collecting process.

24. The information collecting device of claim 23, further comprising a circuit board upon which the controller is mounted, wherein the circuit board is moveable relative to the housing.

25. The information collecting device of claim 24, wherein the device actuator is rigidly fixed to the circuit board.

26. The information collecting device of claim 23, further comprising a switch that is actuated upon activation of the device actuator.

27. The information collecting device of claim 23, further comprising a bottom support that is fixed to the housing.

28. The information collecting device of claim 27, further comprising a circuit board upon which the controller is mounted, wherein, prior to activation of the actuator, the circuit board is spaced from the bottom support.

29. The information collecting device of claim 28, further comprising a spacer being positioned between the bottom support and the circuit board.

30. The information collecting device of claim 29, wherein the spacer comprises a resilient material.

31. The information collecting device of claim 30, wherein the spacer comprises foam.

32. The information collecting device of claim 31, wherein the spacer comprises a spring element.

33. The information collecting device of claim 29, wherein activation of the device actuator compresses the spacer and causes the circuit board to move toward the bottom support.

34. The information collecting device of claim 23, wherein the controller detects occurrence of an activity associated with a fluid receiving device and collects time information associated with the occurrence.

35. The information collecting device of claim 34, wherein the activity comprises entry of fluid into the fluid receiving device.

36. The information collecting device of claim 34, wherein the activity comprises arrival of the fluid receiving device at a physical location.

37. The information collecting device of claim 34, wherein the activity comprises temperature of the fluid passing a threshold temperature.

38. The information collecting device of claim 34, wherein the activity comprises an analyte level of the fluid passing a threshold level.

39. The information collecting device of claim 23, wherein the controller detects a plurality of occurrences of activities associated with a fluid receiving device and collects time information associated with each occurrence.

40. The information collecting device of claim 33, wherein the circuit board includes a switch, and wherein activation of the device actuator compresses the spacer and causes the switch to move toward the bottom support.

41. The information collecting device of claim 40, wherein movement of the switch toward the bottom support causes the switch to contact an actuator of a fluid receiving device, wherein contact between the switch and the actuator of the fluid receiving device triggers the switch and actuates the fluid receiving device to cause fluid to be release from a subject.

42. The information collecting device of claim 23, wherein the controller collects information associated with the fluid receiving device and/or information associated with the fluid over time.

43. The information collecting device of claim 42, wherein the controller begins collecting information when the device actuator is activated.

44. The information collecting device of claim 42, wherein the controller collects information at discrete time intervals.

45. The information collecting device of claim 42, wherein the controller continuously collects information.

46. The information collecting device of claim 23, wherein the housing is removeably attachable to the fluid receiving device.

47. The information collecting device of claim 23, wherein, after the housing is attached to the fluid receiving device, the housing is permanently attached to the fluid receiving device.

48. The information collecting device of claim 23, wherein, after the housing is attached to the fluid receiving device, the housing is limitedly detachable from the fluid receiving device.

49. The information collecting device of claim 23, wherein the fluid receiving device has a needle for penetrating skin of a subject to cause release of fluid from the subject.

50. A method of determining a time at which a fluid receiving portion is actuated to cause fluid to be released from a subject, comprising:
receiving fluid into a fluid receiving device in response to activation of a device actuator;
actuating a controller of an information collecting portion to begin counting time in response to the activation of the device actuator; and
transmitting time information from the controller to a reader,
wherein the information collecting portion and the fluid receiving device are constructed and designed to attach to one another.

51. The method of claim 50, wherein transmitting time information from the controller to a reader comprises sending the time information from the controller to a transponder and sending the time information from the transponder to the reader.

52. The method of claim 51, wherein time information is sent from the transponder to the reader via an antenna.

53. The method of claim 51, wherein time information is sent from the transponder to the reader via wireless communication.

54. The method of claim 51, wherein time information is sent from the transponder to the reader via audio output.

55. The method of claim 50, wherein the information collecting portion is removeably attachable to the fluid receiving portion.

56. The method of claim 50, wherein, after the information collecting portion is attached to the fluid receiving portion, the information collecting portion is permanently attached to the fluid receiving portion.

57. The method of claim 50, wherein, after the information collecting portion is attached to the fluid receiving portion, the information collecting portion is limitedly detachable from the fluid receiving portion.

58. The method of claim 50, further comprising moving a needle of the fluid receiving device relative to a housing of the fluid receiving device to cause fluid to be released from a subject in response to activation of the device actuator.
